(19) United States
(12) Patent Application Publication
(10) Pub. No.: US 2026/0155235 A1
(43) Pub. Date: Jun. 4, 2026

(54) COGNITIVE REHABILITATION TRAINING MULTI-PLAY AND GROUP MANAGEMENT SYSTEM AND METHOD FOR DEMENTIA PREVENTION

(71) Applicant: INTHETECH Inc., Daegu (KR)

(72) Inventor: Seung Ho CHOUN, Daegu (KR)

(21) Appl. No.: 19/405,412

(22) Filed: Dec. 2, 2025

(30) Foreign Application Priority Data
Dec. 2, 2024 (KR) 10-2024-0176556

(51) Int. Cl. G16H 20/70; G16H 15/00; G16H 80/00

(52) U.S. Cl. CPC G16H 20/70; G16H 15/00; G16H 80/00

(57) ABSTRACT

Cognitive rehabilitation training multi-play and group management system and method for dementia prevention disclosed. A cognitive rehabilitation training multi-play and group management system includes a plurality of trainee terminals assigned to a plurality of users grouped into a same group, wherein cognitive rehabilitation training content is executed on each of the trainee terminals, a manager terminal assigned to a manager who manages the same group, configured to receive information regarding an execution status of the cognitive rehabilitation training content executed on the plurality of trainee terminals and display the information on a screen, and a cognitive rehabilitation training management server connected via a network to manage the plurality of trainee terminals and the manager terminal, configured to provide the cognitive rehabilitation training content to each of the plurality of trainee terminals, collect the execution status, and provide information regarding the execution status to the manager terminal.

### STATEMENT REGARDING FEDERALLY SPONSORED RESEARCH OR DEVELOPMENT

This patent is a result of a study conducted with the support of the National IT Industry Promotion Agency (NIPA) (S1301-24-1007, 2024 Gyeongsangnam-do Digital Innovation Hub Creation Support Project Digital Innovation Support (R&BD)_Digital Enterprise in Gyeongnam), funded by the government (Ministry of Science and ICT) in 2024.

### CROSS REFERENCE TO RELATED APPLICATIONS

This patent application claims the priority and benefits of Korean patent application No. 10-2024-0176556, filed on Dec. 2, 2024, which is incorporated herein by reference in its entirety.

### BACKGROUND

The present invention relates to a cognitive rehabilitation training multi-play and group management system and method for dementia prevention.

Recently, as our country has entered a super-aged society, the number of dementia patients has been rapidly increasing. However, depending on the region, some long-term care facilities in the area do not have dedicated dementia care units. Furthermore, dementia-related facilities such as dementia care centers, dementia-specialized hospitals, and day and night care centers for elderly are also insufficient. Therefore, considering the gradually increasing number of dementia patients, it is necessary to establish dementia management services and dementia-related institutions.

In addition, caring for dementia patients requires significant time and expense, making it imperative to introduce efficient solutions that can improve this situation.

A digital cognitive rehabilitation solution is a software-based therapeutic tool designed to enhance and improve cognitive function by consistently and repeatedly training specific cognitive domains that have been particularly impaired due to cognitive disorders.

Even when using a device that provides such a digital cognitive rehabilitation solution, it has been difficult for a single manager to guide and supervise a plurality of therapy subjects (e.g., dementia patients), and there has been a limitation in that, at any given time, only training of the same content and at the same level could be provided to multiple therapy subjects.

The matters described in the background are intended solely to aid in the understanding of the background of the invention and are not necessarily to be construed as prior art already known to those skilled in the art.

### SUMMARY

### Objects of the Invention

The present invention is to provide a cognitive rehabilitation training multi-play and group management system and method for dementia prevention, which enables personalized cognitive rehabilitation training tailored to each therapy subject by providing optimized content based on individual cognitive domains, interests, and levels, even when a single manager manages a plurality of therapy subjects.

The present invention is further to provide a cognitive rehabilitation training multi-play and group management system and method for dementia prevention, which is capable of offering personalized training guidance through real-time analysis of user characteristics and performance patterns, and which includes an artificial intelligence (AI)-based training tutor function that enables real-time error detection and correction.

Other objects of the present invention will become more apparent from the following detailed description.

### Means for Solving the Problems

According to one aspect of the present invention, a cognitive rehabilitation training multi-play and group management system includes a plurality of trainee terminals assigned to a plurality of users grouped into a same group, wherein cognitive rehabilitation training content is executed on each of the trainee terminals, a manager terminal assigned to a manager who manages the same group, configured to receive information regarding an execution status of the cognitive rehabilitation training content executed on the plurality of trainee terminals and display the information on a screen, and a cognitive rehabilitation training management server connected via a network to manage the plurality of trainee terminals and the manager terminal, configured to provide the cognitive rehabilitation training content to each of the plurality of trainee terminals, collect the execution status, and provide information regarding the execution status to the manager terminal, wherein the cognitive rehabilitation training content is divided into group-based training content and personalized training content, and wherein the cognitive rehabilitation training management server is configured to provide the group-based training content collectively to the plurality of trainee terminals during group-based training, and during personalized training, provide the personalized training content to each of the plurality of trainee terminals in a personalized manner corresponding to a status of each of the plurality of users.

The cognitive rehabilitation training management server includes a data integration unit including a central database construction unit, a data standardization unit, and a data analysis unit. The central database construction unit is configured to select and construct a relational database designed with a structure for storing one or more of data regarding the user, therapy records, feedback, and behavioral patterns as a central database. The data standardization unit is configured to unify and standardize a data format stored and managed in the central database, and the data analysis unit is configured to integrate analysis tools and to construct a dashboard that visualizes integrated data by utilizing the analysis tools.

The cognitive rehabilitation training management server may include a user management unit including a group-based training management unit and a personalized management unit, wherein the group-based training management unit groups the plurality of users based on one or more criteria among age, therapy stage, and symptom type to manage them to receive cognitive rehabilitation training on a group basis, and the personalized management unit may create a personalized therapy profile for each of the plurality of users, and a feedback system may be established.

The cognitive rehabilitation training management server includes a user management unit including a group-based training management unit and a personalized management unit, the group-based training management unit is configured to group the plurality of users based on one or more criteria among age, therapy stage, and symptom type to manage them to participate in the cognitive rehabilitation training on a group basis, and the personalized management unit is configured to create a personalized therapy profile for each of the plurality of users, and establish a feedback system.

The cognitive rehabilitation training management server includes an AI training tutor unit that, when a preset content performance pattern such as no response or consecutive incorrect responses is detected during the user's cognitive rehabilitation training performance, is configured to guide a content performance method or a coping measure to promote proper content execution.

The AI training tutor unit is configured to search and invoke a most similar question according to a preset condition for an expected situation during execution of the cognitive rehabilitation training content, and provide an answer from a question-answer dataset as a response by utilizing an embedding model.

The AI training tutor unit is configured to receive a first question from a user, search for a similar question similar to the first question, vectorize the first question and the similar question and calculate a similarity using cosine similarity, and return an answer linked to the similar question with a highest similarity as a response.

According to another aspect of the present invention, cognitive rehabilitation training multi-play and management method performed in a cognitive rehabilitation training multi-play and group management system includes grouping a plurality of users based on one or more criteria among age, therapy stage, and symptom type, providing group-based training content to a plurality of trainee terminals assigned to a plurality of users belonging to a same group and executing the same, creating a personalized therapy profile for each of the plurality of users based on a group training result and providing and executing personalized training content in a personalized manner for each of the plurality of trainee terminals corresponding to the therapy profile.

The method may further include detecting a preset content performance pattern such as no response or consecutive incorrect responses during the user's cognitive rehabilitation training performance and guiding a content performance method or a coping measure to promote proper content execution.

Other aspects, features, and advantages other than those described above will become clear from the following drawings, claims, and detailed description of the invention.

### Effects of the Invention

According to an embodiment of the present invention, it is advantageous to provide individualized cognitive rehabilitation training for dementia prevention by delivering optimized content tailored to each therapy subject's cognitive domain, interest, and level, even when a single manager manages a plurality of therapy subjects.

In addition, it is also advantageous to provide a personalized training guidance through real-time analysis of user characteristics and performance patterns, and to enable real-time error detection and correction by providing an AI-based training tutor function

The effects achievable by the present invention are not limited to those described above, and other effects not explicitly mentioned herein will be readily understood by those skilled in the art from the following description.

### BRIEF DESCRIPTION OF THE DRAWINGS

FIG. 1 illustrates a cognitive rehabilitation training multi-play and management system for dementia prevention according to an embodiment of the present invention;

FIG. 2 is a block diagram showing the configuration of a cognitive rehabilitation training management server included in the cognitive rehabilitation training multi-play and management system according to an embodiment of the present invention;

FIG. 3 illustrates a personalized method managed by the cognitive rehabilitation training management server according to an embodiment compared to a conventional cognitive rehabilitation training management server;

FIG. 4 is a flowchart of a cognitive rehabilitation training multi-play and management method for dementia prevention according to an embodiment of the present invention;

FIG. 5 is a flowchart of an AI-based training guide method performed by an AI training tutor unit according to an embodiment of the present invention;

FIG. 6 is an exemplary view of an AI-based training guide screen executed by the AI training tutor unit;

FIG. 7 is exemplary screens of contents provided by a content providing unit; and

FIG. 8 illustrates a configuration of a system according to an embodiment of the present invention.

### DETAILED DESCRIPTION OF THE EMBODIMENTS

The invention can be modified in various forms and specific embodiments will be described below and illustrated with accompanying drawings. However, the embodiments are not intended to limit the invention, but it should be understood that the invention includes all modifications, equivalents, and replacements belonging to the concept and the technical scope of the invention.

When an element is referred to as being “connected to” or “coupled to” another element, it should be understood that still another element may be interposed therebetween, as well as that the element may be connected or coupled directly to another element. On the contrary, if it is mentioned that an element is “connected directly to” or “coupled directly to” another element, it should be understood that still another element is not interposed therebetween.

Terms such as first, second, etc., may be used to refer to various elements, but, these element should not be limited due to these terms. These terms will be used to distinguish one element from another element.

The terms used in this specification are intended to merely describe specific embodiments, but not intended to limit the invention. An expression of the singular number includes an expression of the plural number, so long as it is clearly read differently. The terms such as “include” and “have” are intended to indicate that features, numbers, steps, operations, elements, components, or combinations thereof used in the following description exist and it should thus be understood that the possibility of existence or addition of one or more other different features, numbers, steps, operations, elements, components, or combinations thereof is not excluded.

In addition, elements of an embodiment described below with reference to the accompanying drawings are not limited to the corresponding embodiment, may be included in another embodiment without departing from the technical spirit of the invention. Although particular description is not made, plural embodiments may be embodied as one embodiment.

In describing the invention with reference to the accompanying drawings, like elements are referenced by like reference numerals or signs regardless of the drawing numbers and description thereof is not repeated. If it is determined that detailed description of known techniques involved in the invention makes the gist of the invention obscure, the detailed description thereof will not be made.

In addition, the terms such as “. . . part”, “. . . unit”, “. . . module”, “. . . device”, etc. described in the specification mean a unit that processes at least one function or operation, and this can be implemented as a combination of hardware, software, or hardware and software.

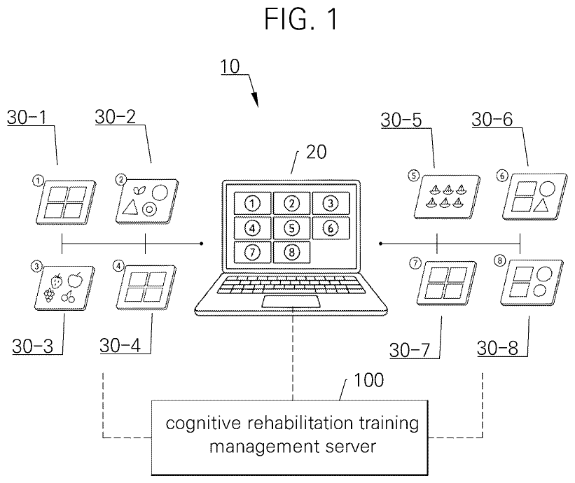

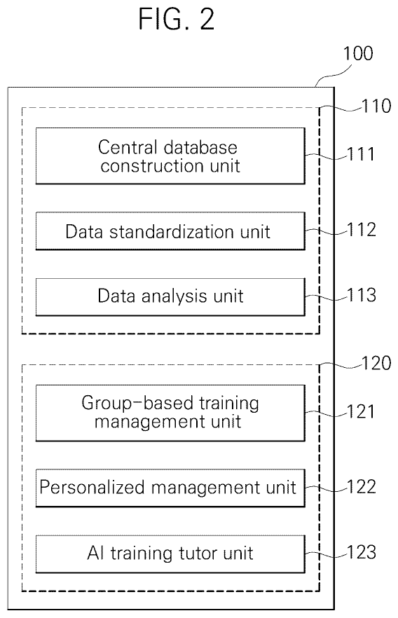

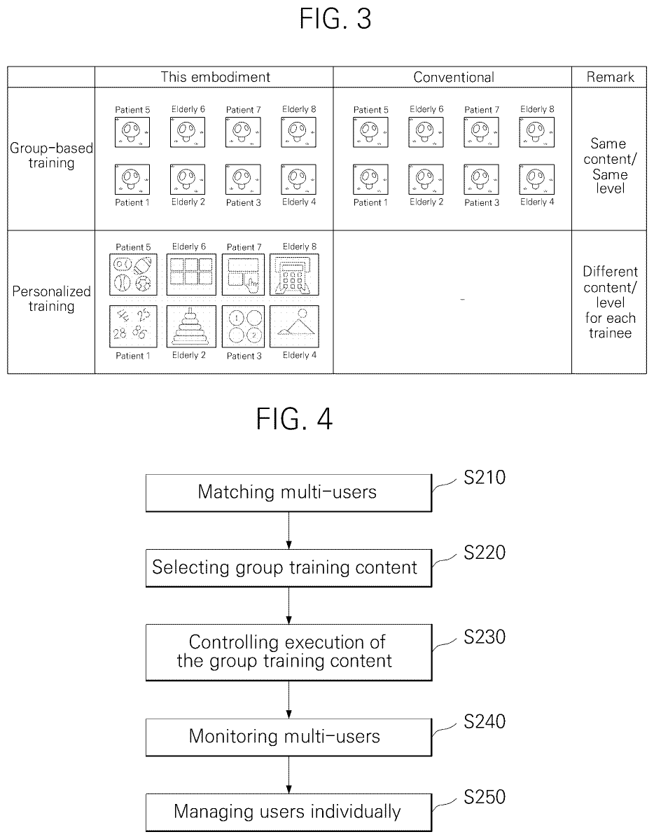

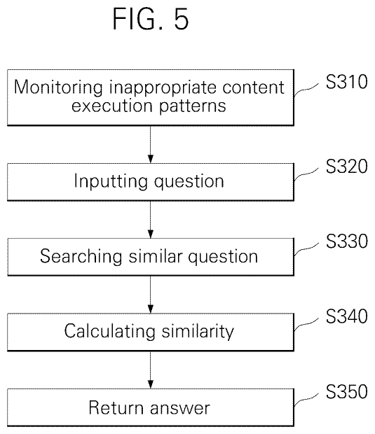

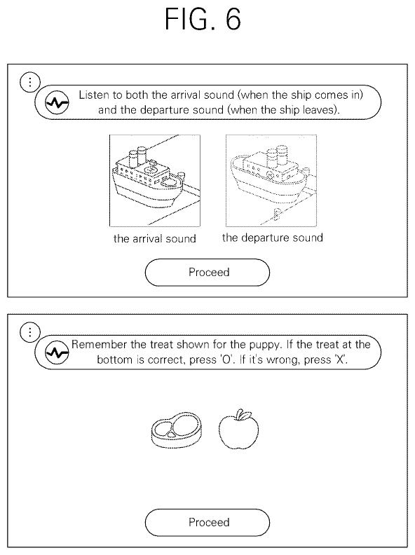

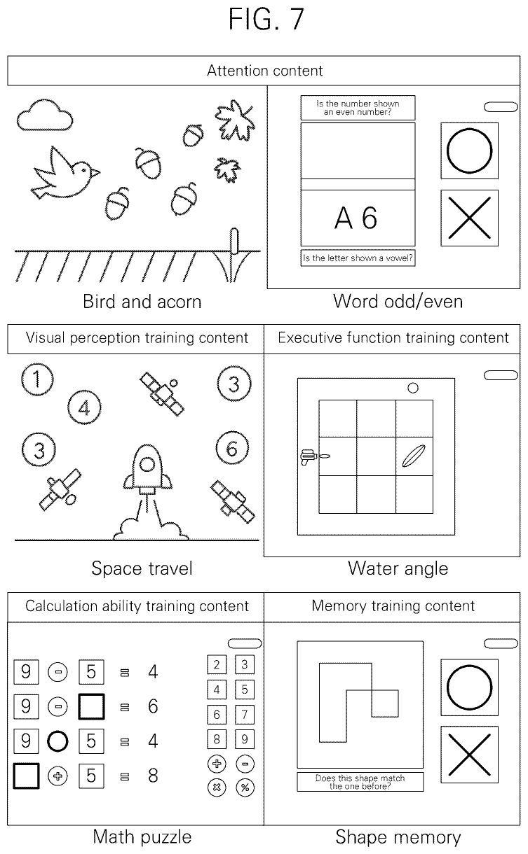

FIG. 1 illustrates a cognitive rehabilitation training multi-play and management system for dementia prevention according to an embodiment of the present invention, FIG. 2 is a block diagram showing the configuration of a cognitive rehabilitation training management server included in the cognitive rehabilitation training multi-play and management system according to an embodiment of the present invention, FIG. 3 illustrates a personalized method managed by the cognitive rehabilitation training management server according to an embodiment compared to a conventional cognitive rehabilitation training management server, FIG. 4 is a flowchart of a cognitive rehabilitation training multi-play and management method for dementia prevention according to an embodiment of the present invention, FIG. 5 is a flowchart of an AI-based training guide method performed by an AI training tutor unit according to an embodiment of the present invention, FIG. 6 is an exemplary view of an AI-based training guide screen executed by the AI training tutor unit, and FIG. 7 is exemplary screens of contents provided by a content providing unit.

A cognitive rehabilitation training multi-play and management system and method for dementia prevention according to an embodiment of the present invention is characterized in that it enables personalized cognitive rehabilitation training for dementia prevention by providing optimized content tailored to an individual's cognitive domain, interests, level, etc., for a plurality of rehabilitation subjects managed by a single manager.

Referring to FIG. 1, a cognitive rehabilitation training multi-play and management system 10 according to the present embodiment may include a cognitive rehabilitation training management server 100, a manager terminal 20, and a plurality of trainee terminals 30-1 to 30-8 (hereinafter collectively referred to as ‘30’). The cognitive rehabilitation training management server 100, the manager terminal 20, and the plurality of trainee terminals 30 may be configured to communicate with each other via wired or wireless networks.

The manager terminal 20 is a terminal device operated by a manager who manages a plurality of trainees (rehabilitation subjects or dementia patients). The manager terminal 20 is a computing device capable of accessing a web page through a web browser or downloading and installing an application, and may be, for example, a smartphone, a tablet PC, a laptop, a desktop PC.

The trainee terminal 30 is a terminal device operated by a trainee (user, rehabilitation subject, etc.) who performs cognitive rehabilitation training for dementia prevention. The trainee terminal 30 is a computing device capable of accessing a web page through a web browser or downloading and installing an application, and may be, for example, a smartphone, a tablet PC, a laptop, a desktop PC.

The cognitive rehabilitation training management server 100 stores and manages content for cognitive rehabilitation training for dementia prevention, provides such content to the plurality of trainee terminals 30, and provides a training status to the manager terminal 20.

The cognitive rehabilitation training management server 100 may operate as a group-based cognitive rehabilitation training program, enabling simultaneous monitoring and training of the entire trainee group, and may provide both group-based training and individualized training tailored to each user. In addition, it supports a manager's quick decision-making through real-time monitoring and pattern analysis. Compared to conventional cognitive rehabilitation training, the system enables objective observation of therapeutic progress and immediate feedback to the trainees, thereby enhancing rehabilitation motivation and the perceived importance of training. In addition, it is possible to reduce the amount of time spent on direct training guidance by the manager (therapist) and facilitate the acquisition of objective data.

Referring to FIG. 2, the cognitive rehabilitation training management server 100 may include a data integration unit 110 and a user management unit 120.

The data integration unit 110 may include a central database construction unit 111, a data standardization unit 112, and a data analysis unit 113.

The central database construction unit 111 may be configured to design a structure that stores user data, therapy records, feedback, and behavioral patterns by selecting a relational database as the central database. The database may be scalable and capable of accommodating various data types.

In addition, the central database construction unit 111 may be configured to implement an ETL process to extract, transform, and load data from existing individual systems. This process helps maintain data consistency and eliminate duplicate data for integration into the central database.

The data standardization unit 112 may be configured to unify the format of data stored and managed in the central database. It may standardize the formats of data collected from various systems to maintain a consistent data structure. For example, it may define data schemas and set verification rules for data input.

Furthermore, the data standardization unit 112 may be configured to perform metadata management by recording the source, creation time, and conversion process of the data to enhance data reliability and ensure transparency in data management. A metadata management tool may be used to continuously monitor data quality.

The data analysis unit 113 may be configured to integrate analytical tools and construct a dashboard that visualizes and analyzes the integrated data. Through this dashboard, user behavior, therapeutic outcomes, and feedback may be monitored in real time. The monitoring results may also be shared with the manager terminal 20 to allow the manager to review them.

Additionally, the data analysis unit 113 may be configured to automatically generate reports on user behavior and therapeutic outcomes on a regular basis. These automatically generated reports may be shared within the manager team and used to support decision-making. Report templates may be preset to ensure consistent formatting of the data.

The user management unit 120 may include a group-based training management unit 121 and a personalized management unit 122.

The group-based training management unit 121 may be configured to manage users by grouping them to participate in cognitive rehabilitation training on a group basis. Users may be grouped based on various criteria such as age, therapy stage, or symptom type. Tailored therapy plans may be established for specific groups, and analysis and feedback may be provided based on the grouped data.

Moreover, the group-based training management unit 121 may include a manager dashboard function. As illustrated in FIG. 1, the manager dashboard may be provided to the manager terminal 20 and displayed on the terminal screen, allowing the manager to monitor the progress of each group and intervene with individual users as needed. The dashboard may visually present the cognitive rehabilitation training content being performed by each group, therapeutic outcomes, user participation levels, and feedback, enabling intuitive understanding.

Referring to FIG. 3, a comparative table is illustrated showing the differences between conventional methods and the group-based and personalized management functions according to the present embodiment.

Unlike conventional approaches in which a single management server provides identical content to multiple rehabilitation subjects using remote functionality, optimized content tailored to each individual's cognitive domain, interest, and level can be provided. Training effects can be maximized by providing optimized content that reflects an individual's cognitive ability level.

In addition, personalized content can be provided through real-time analysis of user's characteristics and performance patterns.

Referring to FIG. 4, the group-based training management unit 121 matches multi-users (Step S210). The multi-users may be a plurality of users grouped according to a preset criterion.

Group training may be initiated by selecting group training content for the multi-users (Step S220) and controlling execution of the group training content (Step S230).

After executing the group training content, a terminal status and a training status for each rehabilitation subject may be monitored (Step S240), and then stored in each rehabilitation subject's database to be managed individually (Step S250). Through the personalized management, content usage frequency, training progress graphs for each cognitive domain, etc. can be provided.

Next, the personalized management unit 122 may be configured to create a personalized therapy profile for each user. Based on each user's health status, therapy history, and preferences, the personalized therapy profile may be created, which can be used to propose an optimized therapy plan tailored to the user.

In addition, the personalized management unit 122 may be configured to establish a feedback system that allows users to easily communicate their experiences or discomforts felt during a therapy process. Through this, opinions can be collected and reflected in system improvements.

Additionally, the cognitive rehabilitation training management server 100 according to the present embodiment may further include an AI training tutor unit 123. The AI training tutor unit 123 may guide the user toward proper content execution when persistent inappropriate content interaction patterns are detected during cognitive rehabilitation training.

For example, in content with a time limit, a notification may be provided when one-third of the time remains, and if there is no response for a preset duration (e.g., 5 seconds) or more, or if three consecutive incorrect responses occur, guidance on how to proceed or a coping measure may be provided via a help button.

The AI training tutor unit 123 may is configured to provide real-time feedback to a user. It can continuously improve the accuracy of the AI training tutor function by collecting and analyzing user data.

The AI training tutor unit 123 may operate as a context-based automatic response system. For an expected situation during use of cognitive rehabilitation training content, it may identify appropriate questions based on preset conditions and utilize an embedding model to retrieve the most suitable response from a question-answer dataset.

For instance, if a user fails to complete a specific step during content execution, the AI training tutor unit 123 may calculate semantic similarity between the current condition and preconfigured questions to provide appropriate guidance.

The AI training tutor unit 123 may calculate a semantic similarity between a question called under a specific condition and a question-answer dataset using an embedding model (bge-base-en-v1.5). Through this, it is possible to evaluate similarity based on context and meaning beyond simple keyword matching, and to provide a most appropriate response. The embedding model converts data into a vector. And a similarity calculation between a user input question and prestored questions can be performed using cosine similarity.

Referring to FIG. 5, a flowchart of an AI training tutor method performed by the AI training tutor unit 123 is shown.

While monitoring of the user's content execution process, inappropriate content execution patterns may be detected (Step S310). Such patterns may include lack of response or consecutive incorrect responses.

In this case, the user inputs a question (Step S320). The question may be input to an Ask(string question) function.

The AI training tutor unit 123 searches for a similar question (Step S330). A search. Search(question, 1) function retrieves the most similar question to the input. Here, the parameter “1” indicates that only the single most similar question should be returned. A similarity calculation algorithm is applied within this function.

Next, a similarity is calculated (Step S340). The search. Search function calculates a similarity between the input question and stored questions. This is based on embedding-based search, in which the question is vectorized and cosine similarity is used to identify the nearest vector (Approximate Nearest Neighbor search). Cosine similarity evaluates similarity by measuring an angle between each vector after text vectorization.

After similarity is calculated, the most similar question is identified, and a response linked to the corresponding question is returned (Step S350). The response return may be performed in dialogues[similarQuestion].

FIG. 6 illustrates a cognitive rehabilitation training guide screen according to the AI training tutor method performed by the AI training tutor unit 123.

As described above, the AI training tutor unit 123 may perform a search based on semantic similarity. It calculates a similarity between a question and an answer by utilizing an AI embedding model capable of understanding the meaning and context of a sentence. For example, even if two sentences, “What should I do when I haven't clicked the proceed button?” and “How can I start the session?”, are phrased differently, they can be understood as having the same meaning to provide an appropriate answer.

In addition, the AI training tutor unit 123 may invoke a suitable question based on situational awareness. When a user encounters a specific situation (e.g., failing to press a button or repeatedly submitting incorrect responses), it invokes a suitable question according to a preset condition and provides assistance tailored to the user's needs.

This AI training tutor unit 123 may operate as an efficient learning assistant, and can maximize a learning effect according to cognitive rehabilitation training by providing appropriate help when a user is unsure of what action to take or repeatedly makes mistakes during content execution.

The AI training tutor unit 123 functions as a learning assistant. Within learning content (cognitive rehabilitation training content), it provides additional guidance according to a preset condition in line with a user's behavior. This approach is designed not merely to observe user's behavior but to actively support improved learning outcomes.

In addition, it provides predictable and consistent guidance. Since the system provides elaborately prepared answers based on predictable questions in a specific situation, it can prevent a user from experiencing unnecessary confusion while progressing through content.

In addition, it can also provide help at appropriate moments without requiring the user to input a question. This makes it possible to create a personalized learning experience similar to a real tutor observing and advising the user throughout the training process.

FIG. 7 illustrates the cognitive rehabilitation training content provided to users in the system according to the present embodiment. Based on cognitive science, the content is designed to train various cognitive abilities including attention, visual perception, executive function, calculation ability, memory, and orientation. Attention relates to sustained attention, selective attention, and arousal; memory relates to working memory, recall, and recognition; orientation relates to person, space, and time; calculation ability relates to number comprehension and language function; visual perception relates to visual information and information integration; and executive function relates to planning, problem solving, and judgment.

The bird and acorn content, which is attention content shown in FIG. 7, is content that makes a bird fly by tapping a screen and prevents the bird from falling to the ground or touching obstacles other than acorns. The word odd/even content, which is other attention content, is content for selecting an O or X button according to a presented problem.

The space travel content, which is visual perception training content, is content for moving a planet in numerical order or in numerical→alphabetical order by touching a spaceship.

The water angle content, which is executive function training content, is content for calculating an angle of a presented surfboard and selecting a position where a water droplet will reach.

The math puzzle content, which is calculation ability training content, is content for touching and dragging a number or a symbol that fits a formula.

The shape memory content, which is memory training content, is content for selecting an O or X button according to a problem by comparing a color or a shape of a previously presented figure and a currently presented figure.

According to the cognitive rehabilitation training multi-play and group management system and method for dementia prevention of the present embodiment, an artificial intelligence (AI)-based technology is introduced, and personalized training can be provided by implementing an AI assistant function. A user pattern analysis and prediction model using a machine learning algorithm may be developed and utilized.

Additionally, the enhancement of the group training manager system improves operational efficiency, and real-time monitoring and data analysis capabilities may be advanced.

Various cognitive domains can be covered through new content. Customized training can be supported through content configuration by difficulty level and by topic. Program design reflecting the latest cognitive rehabilitation research results may be applied to these contents.

In addition, user's experience can be optimized. An intuitive and user-friendly interface can be applied. User satisfaction can be improved by providing a personalized training program. Training effects can be maximized by establishing a real-time feedback system.

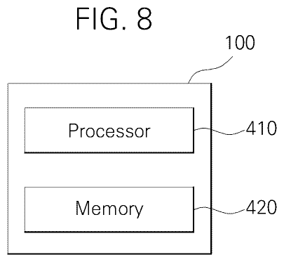

FIG. 8 illustrates a configuration of a system according to an embodiment of the present invention.

Referring to FIG. 8, the cognitive rehabilitation training multi-play and group management system 10 and/or the cognitive rehabilitation training management server 100 may include a processor 410 and a memory 420. The memory 420 stores one or more instructions executable by the processor 410. The processor 410 executes the one or more instructions stored in the memory 420. The processor 410 may execute one or more operations described above in connection with FIGS. 3 to 7 by executing an instruction. In addition, the configuration of the present invention described above with reference to FIG. 2 may be a configuration implemented by an instruction executed by the processor 410.

The embodiments described above may be implemented as a hardware component, a software component, and/or a combination of a hardware component and a software component. For example, the devices, methods, and elements described in the embodiments may be implemented using one or more general-purpose computers or special-purpose computers, such as a processor, a controller, a Central Processing Unit (CPU), a Graphics Processing Unit (GPU), an arithmetic logic unit (ALU), a digital signal processor, a microcomputer, a field programmable gate array (FPGA), a programmable logic unit (PLU), a microprocessor, Application Specific Integrated Circuits (ASICs), or any other device capable of executing and responding to an instruction.

The aforementioned cognitive rehabilitation training multi-play and group management method may also be implemented in the form of a recording medium including a computer-executable instruction such as an application or a program module executed by a computer. A computer-readable medium may be any available medium that can be accessed by a computer and includes both volatile and non-volatile media, and removable and non-removable media. In addition, a computer-readable medium may include a computer storage medium. A computer storage medium includes both volatile and non-volatile, removable and non-removable media implemented in any method or technology for storage of information such as computer-readable instructions, data structures, program modules, or other data.

The aforementioned cognitive rehabilitation training multi-play and group management method may be executed by an application basically installed on a terminal (which may include a program included in a platform or an operating system basically mounted on the terminal), and may also be executed by an application (i.e., a program) directly installed on a master terminal by a user through an application providing server such as an application store server, an application, or a web server related to the corresponding service. In this sense, the aforementioned cognitive rehabilitation training multi-play and group management method may be implemented as an application (i.e., a program) basically installed on a terminal or directly installed by a user and recorded on a computer-readable recording medium such as a terminal.

Although the embodiments of the present invention have been described with reference to the above, it will be understood by those with ordinary skill in the art that the present invention can be variously modified and changed without departing from the spirit and scope of the present invention described in the following claims.

What is claimed is:

1. A cognitive rehabilitation training multi-play and group management system, comprising:
a plurality of trainee terminals assigned to a plurality of users grouped into a same group, wherein cognitive rehabilitation training content is executed on each of the trainee terminals;
a manager terminal assigned to a manager who manages the same group, configured to receive information regarding an execution status of the cognitive rehabilitation training content executed on the plurality of trainee terminals and display the information on a screen; and
a cognitive rehabilitation training management server connected via a network to manage the plurality of trainee terminals and the manager terminal, configured to provide the cognitive rehabilitation training content to each of the plurality of trainee terminals, collect the execution status, and provide information regarding the execution status to the manager terminal,
wherein the cognitive rehabilitation training content is divided into group-based training content and personalized training content, and
wherein the cognitive rehabilitation training management server is configured to provide the group-based training content collectively to the plurality of trainee terminals during group-based training, and during personalized training, provide the personalized training content to each of the plurality of trainee terminals in a personalized manner corresponding to a status of each of the plurality of users.

2. The cognitive rehabilitation training multi-play and group management system of claim 1, wherein the cognitive rehabilitation training management server comprises a data integration unit including a central database construction unit, a data standardization unit, and a data analysis unit,
wherein the central database construction unit is configured to select and construct a relational database designed with a structure for storing one or more of data regarding the user, therapy records, feedback, and behavioral patterns as a central database,
wherein the data standardization unit is configured to unify and standardize a data format stored and managed in the central database, and
wherein the data analysis unit is configured to integrate analysis tools and to construct a dashboard that visualizes integrated data by utilizing the analysis tools.

3. The cognitive rehabilitation training multi-play and group management system of claim 1, wherein the cognitive rehabilitation training management server comprises a user management unit including a group-based training management unit and a personalized management unit,
wherein the group-based training management unit is configured to group the plurality of users based on one or more criteria among age, therapy stage, and symptom type to manage them to participate in the cognitive rehabilitation training on a group basis, and
wherein the personalized management unit is configured to create a personalized therapy profile for each of the plurality of users, and establish a feedback system.

4. The cognitive rehabilitation training multi-play and group management system of claim 1, wherein the cognitive rehabilitation training management server comprises an AI training tutor unit that, when a preset content performance pattern such as no response or consecutive incorrect responses is detected during the user's cognitive rehabilitation training performance, is configured to guide a content performance method or a coping measure to promote proper content execution.

5. The cognitive rehabilitation training multi-play and group management system of claim 4, wherein the AI training tutor unit is configured to search and invoke a most similar question according to a preset condition for an expected situation during execution of the cognitive rehabilitation training content, and provide an answer from a question-answer dataset as a response by utilizing an embedding model.

6. The cognitive rehabilitation training multi-play and group management system of claim 5, wherein the AI training tutor unit is configured to receive a first question from a user, search for a similar question similar to the first question, vectorize the first question and the similar question and calculate a similarity using cosine similarity, and return an answer linked to the similar question with a highest similarity as a response.

7. A cognitive rehabilitation training multi-play and management method performed in a cognitive rehabilitation training multi-play and group management system, comprising:
grouping a plurality of users based on one or more criteria among age, therapy stage, and symptom type;
providing group-based training content to a plurality of trainee terminals assigned to a plurality of users belonging to a same group and executing the same;
creating a personalized therapy profile for each of the plurality of users based on a group training result; and
providing and executing personalized training content in a personalized manner for each of the plurality of trainee terminals corresponding to the therapy profile.

8. The cognitive rehabilitation training multi-play and management method of claim 7 further comprising:
detecting a preset content performance pattern such as no response or consecutive incorrect responses during the user's cognitive rehabilitation training performance; and
guiding a content performance method or a coping measure to promote proper content execution.
